(19) United States
(12) Patent Application Publication
(10) Pub. No.: US 2016/0338717 A1
(43) Pub. Date: Nov. 24, 2016

(54) Method and Implement for Increasing Vascularization

(71) Applicant: Robert E. Frank, Oak Park, IL (US)

(72) Inventor: Robert E. Frank, Oak Park, IL (US)

(21) Appl. No.: 15/156,815

(22) Filed: May 17, 2016

Related U.S. Application Data
(60) Provisional application No. 62/164,913, filed on May 21, 2015.

(51) Int. Cl. A61B 17/322

(52) U.S. Cl. CPC A61B 17/322; A61B 2017/0023

(57) ABSTRACT

An implement for increasing tissue vascularization includes a handle with a receiving post and a retainer movable between a first position and a second position, and a cutting wheel disposed on the receiving post. The cutting wheel includes at least one cutting element disposed on an outer periphery of the cutting wheel. In the first position, the handle permits the cutting wheel to be mounted on the receiving post. In the second position, the cutting wheel is held in place with respect to the receiving post yet freely rotatable with respect to the receiving post.

### CROSS-REFERENCE TO PRIOR APPLICATION

Priority is claimed to U.S. Provisional Patent Application No. 62/164,913, filed on May 21, 2015, the entire disclosure of which is incorporated by reference herein.

### FIELD

The present invention relates to a method and implement for increasing the vascularization of tissue, for example, in the human body.

### BACKGROUND

Surgical procedures may cause scar tissue to develop on or in the body of a surgical patient. In the context of graft material, tissue is placed in the patient's body for cosmetic or reconstructive purposes. If the graft material is not revascularized, the likelihood of the patient's body accepting the graft material may decrease. Graft material may include tissue from elsewhere in the patient's own body (i.e., autologous tissue), including skin grafts, tendon grafts, and bone grafts, and artificial tissues such as allografts and xenografts, both of which are commercially available.

A well vascularized tissue bed is helpful for both the integration of the graft material and the delivery of growth factors of medications. However, placing growth factors, chemotherapeutic agents, or graft material onto scar tissue, for example, in the case of a breast capsule, can make absorption of growth agents and revascularization difficult.

Options for making vascular channels at the recipient site accessible include removal of scar tissue via, in the case of the breast, either capsullectomy or capsullorhaphy, or manual fenestration of the wound bed. Removal of scar tissue thins native tissues, which may already be attenuated. Scoring scar tissue, or capsullorhaphy, is most often done using electric cautery, which effectively seals the vascular channels and adds a layer of char to the wound bed. Manual fenestration, which involves dimpling or piercing the wound bed using a scalpel or similar device, has been demonstrated to increase the rate of revascularization, but is tedious and imprecise and may result in injury to structures immediately below the scar layer.

### SUMMARY

Using an implement as described, discrete incisions of a controlled depth are created within the scarred wound bed. Use of this implement results in exposure of vascular channels without the use of cautery and may also aid in absorption of topical medications and growth factors, which require open vascular channels for absorption.

An implement for increasing tissue vascularization includes a handle with a receiving post and a retainer movable between a first position and a second position, and a cutting wheel disposed on the receiving post. The cutting wheel includes at least one cutting element disposed on an outer periphery of the cutting wheel. In the first position, the handle permits the cutting wheel to be mounted on the receiving post. In the second position, the cutting wheel is held in place with respect to the receiving post yet freely rotatable with respect to the receiving post.

### BRIEF DESCRIPTION OF THE DRAWINGS

The present invention will be described in greater detail below based on the exemplary figures. The invention is not limited to the exemplary embodiments. Other features and advantages of various embodiments of the present invention will become apparent by reading the following detailed description with reference to the attached drawings, which illustrate the following:

FIG. 1 is a perspective view of an implement for increasing vascularization;

FIG. 2 is a perspective view of the implement of FIG. 1 with a retainer in a first position and without a cutting wheel;

FIG. 3 is a perspective view of the implement of FIG. 1 with a retainer in a first position and with a cutting wheel;

FIG. 4 is a perspective view of the implement of FIG. 1 with a retainer in a second position and without a cutting wheel;

FIG. 5 is a front view of a first embodiment of a cutting wheel;

FIG. 6 is a side view of the cutting wheel of FIG. 5;

FIG. 7 is a front view of a second embodiment of a cutting wheel;

FIG. 8 is a side view of the cutting wheel of FIG. 7;

FIG. 9 is a front view of a third embodiment of a cutting wheel;

FIG. 10 is a side view of the cutting wheel of FIG. 9;

FIG. 11 is a front view of a fourth embodiment of a cutting wheel;

FIG. 12 is a front view of a fifth embodiment of a cutting wheel; and

FIG. 13 is a side view of the cutting wheel of FIG. 12.

### DETAILED DESCRIPTION

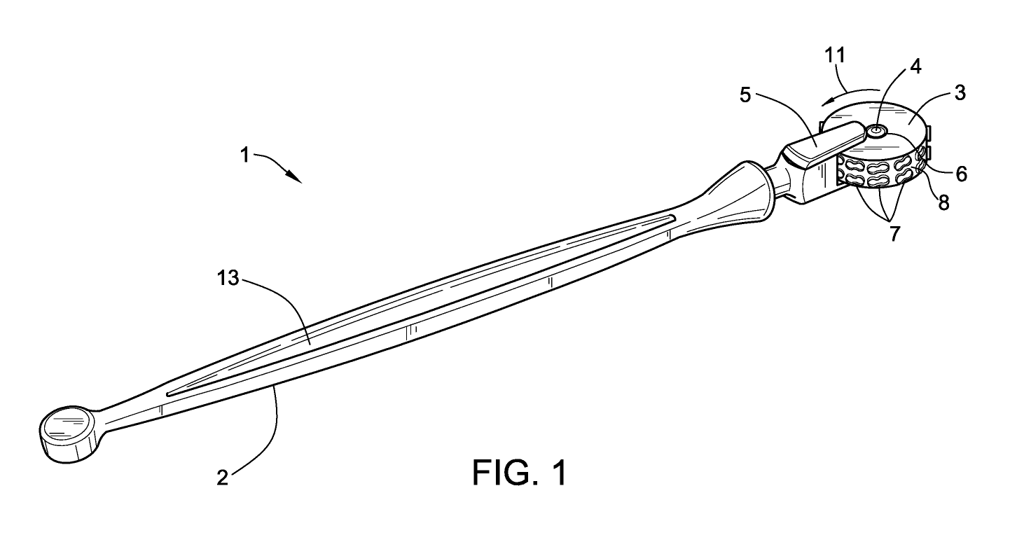

An implement 1 for increasing vascularization in tissue, such as scar tissue, includes a handle 2 and a cutting wheel 3. The cutting wheel 3 is mountable on a receiving post 4 of the handle 2 where it is held in place by a retainer 5 yet freely rotatable with respect to receiving post 4. The components of implement 1 are shown in FIGS. 1-13.

The cutting wheel 3 includes a bore 6 at its center. The shape of bore 6 is complementary to the shape of receiving post 4. For example, receiving post 4 and bore 6 may be cylindrical in shape.

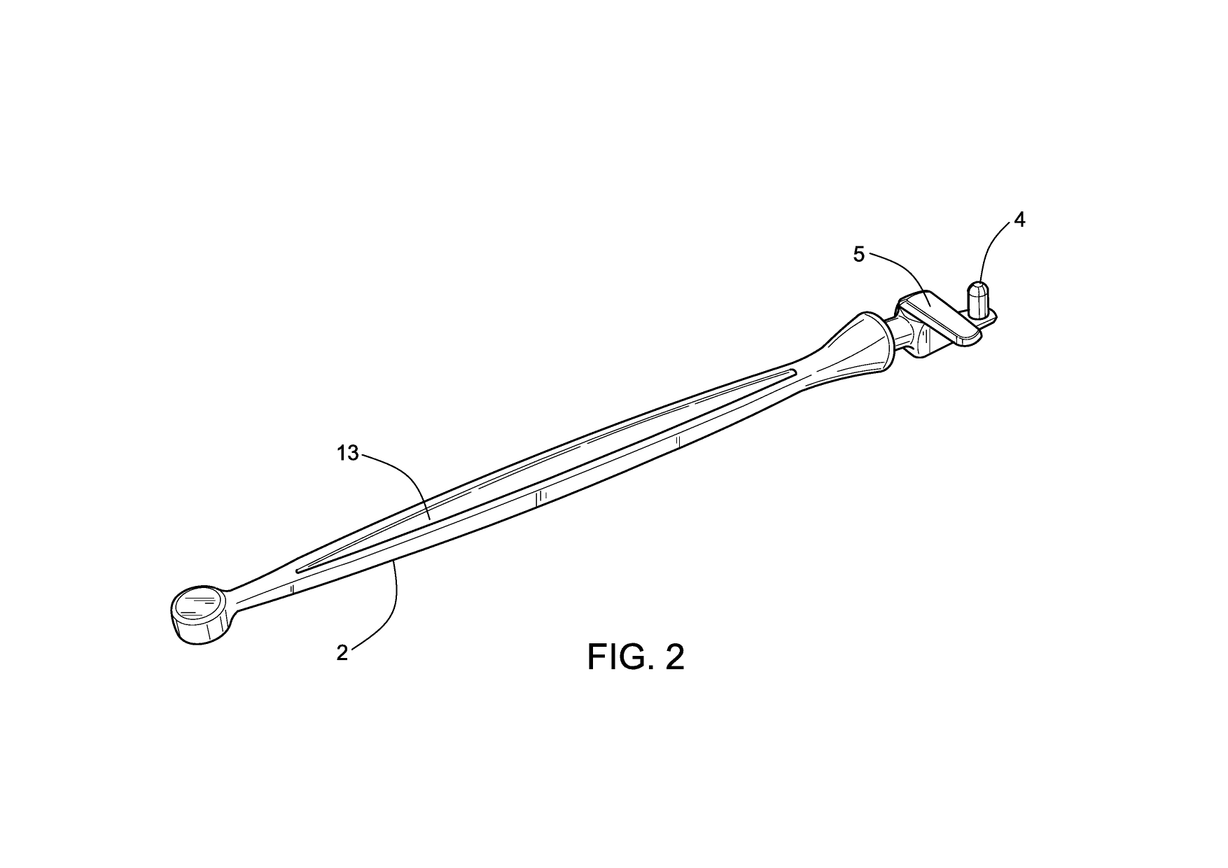

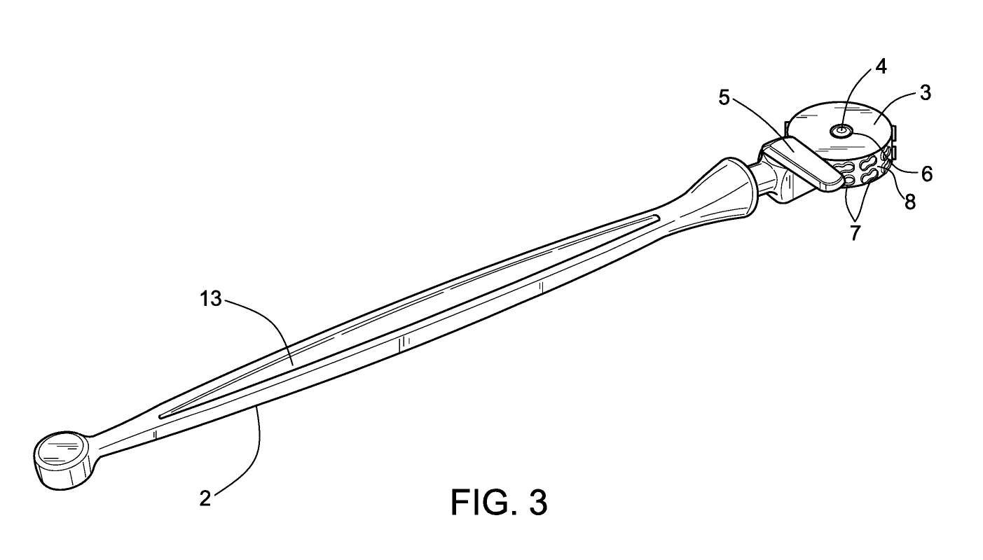

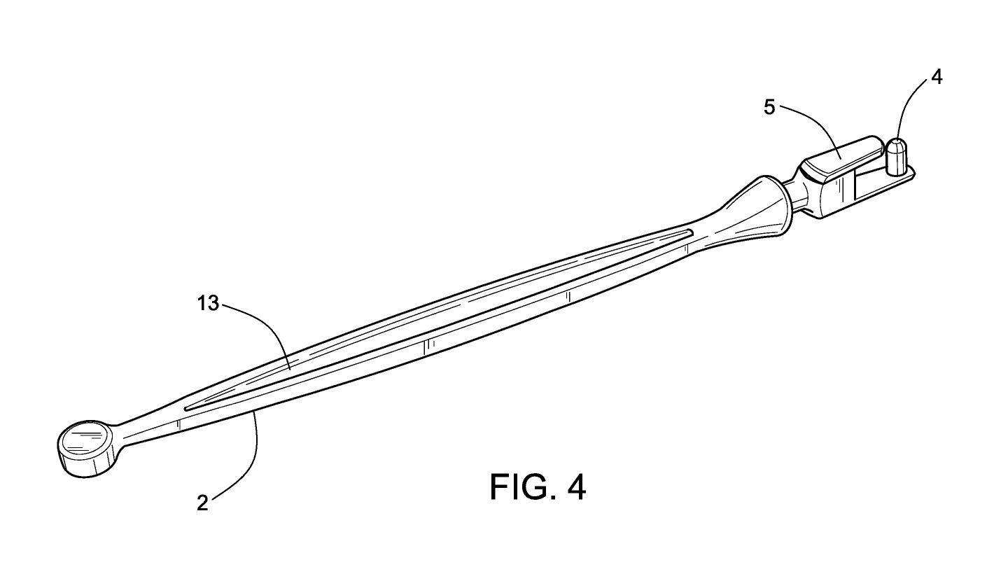

Retainer 5 is configured to be moveable with respect to the handle 2 between at least a first position and a second position. When retainer 5 is in the first position, as shown in FIGS. 2-3, retainer 5 does not impede the insertion of receiving post 4 into bore 6 of cutting wheel 3, and cutting wheel 3 can be mounted on receiving post 4. FIG. 3 shows cutting wheel 3 mounted on receiving post 4 of handle 2 with retainer 5 in the first position. After cutting wheel 3 is mounted on receiving post 4, retainer 5 is moved from the first position to the second position. In the second position, retainer 5 holds cutting wheel 3 in place with respect to receiving post 4 such that the cutting wheel 3 does not disengage from receiving post 4. FIG. 1 shows cutting wheel 3 mounted on receiving post 4 with retainer 5 in the second position. For illustrative purposes, FIG. 4 shows retainer 5 in the second position when cutting wheel 3 is not mounted on receiving post 4. The configuration of implement 1 shown in FIG. 4 may, for example, be used during the shipping or storing of handle 2. In the first position, as shown in FIGS. 2-3, retainer 5 may be orthogonal to a longitudinal axis of handle 2. In the second position, as shown in FIGS. 1 and 4, retainer 5 may be collinear with the longitudinal axis of handle 2.

Referring again to FIG. 1, cutting wheel 3 includes at least one cutting element 7 disposed on an outer periphery 8 of cutting wheel 3. Cutting wheel 3 preferably includes a plurality of cutting elements 7 disposed on outer periphery 8.

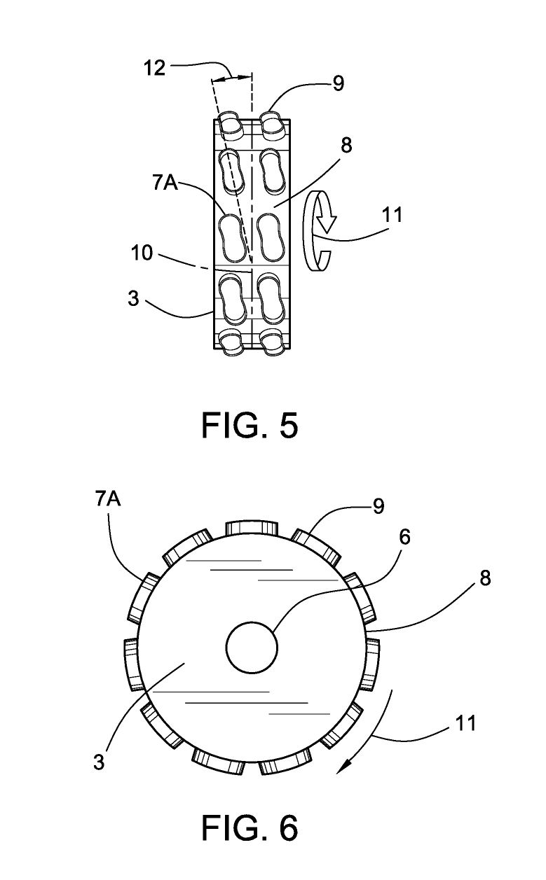

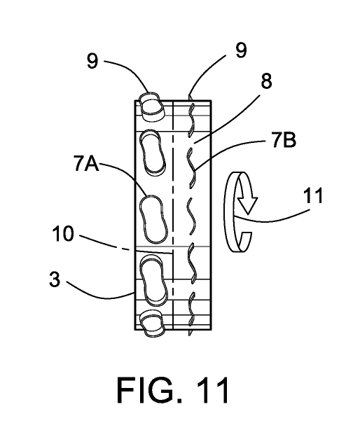

FIGS. 5-13 show example variants of cutting element 7 identified as cutting elements 7A, 7B, 7C, and 7D. For simplicity, however, cutting elements 7A, 7B, 7C, and 7D are often discussed collectively as cutting elements 7. It is contemplated that cutting wheel 3 may include a variety of different cutting elements 7, for example, some combination of one or more of cutting elements 7A, 7B, 7C, and 7D, or any other cutting elements 7. FIG. 11 shows a cutting wheel 3 with a first row of cutting elements 7A disposed on one side of a center line circumference 10 of the cutting wheel 3 and a second row of cutting elements 7B disposed on the other side of center line circumference 10.

Cutting elements 7 generally protrude radially outward from outer periphery 8 of cutting wheel 3. Each cutting element 7 includes a cutting edge 9, which is used for perforating or cutting tissue or the like, for example to increase vascularization of the tissue, during rotation of cutting wheel 7 when it is mounted on handle 2, as shown in FIG. 1. The height of a cutting element 7 is the distance from the outer periphery 8 of cutting wheel 3 to the cutting edge 9 of the cutting element 7. The height of a cutting element 7 may be, for example, from approximately 1 to 5 mm, and is preferably 4.1 mm. In another embodiment, the height of a cutting element 7 ranges from 2 to 4 mm.

The cutting elements 7 may be of a variety of shapes. As shown in FIGS. 5-6, cutting element 7A may be a closed shape, meaning that the cutting edge 9 of cutting element 7A closes back on itself. Such closed shapes are particularly useful for excising pieces of tissue from the area where vascularization is sought to be increased. The closed shape may be oblong, with a width of the cutting element 7A being shorter than a length of the cutting element 7A. Cutting element 7A may be characterized as jelly bean-shaped or peanut-shaped. When cutting element 7 is uniquely shaped, as in cutting element 7A for example, the user of the implement 1 can more easily visualize the tissue over which the cutting element 7 has already traveled, promoting efficiency and decreasing the likelihood that a given piece of tissue is cut or perforated more than necessary.

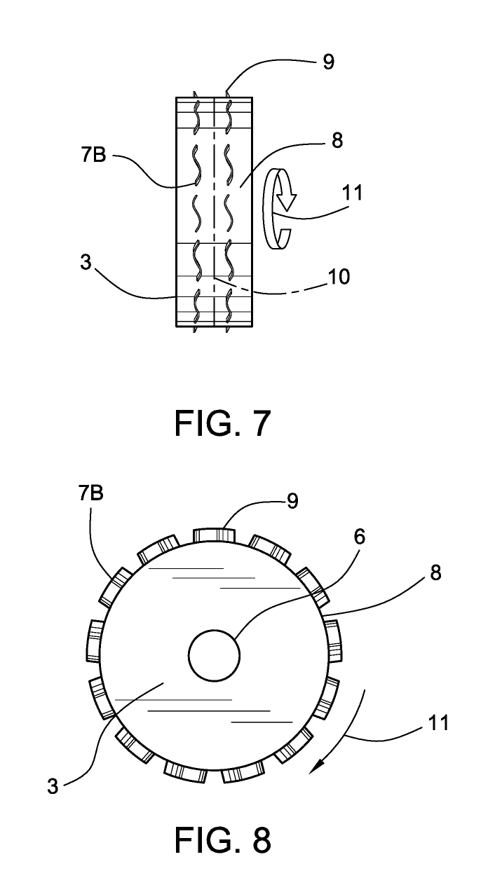

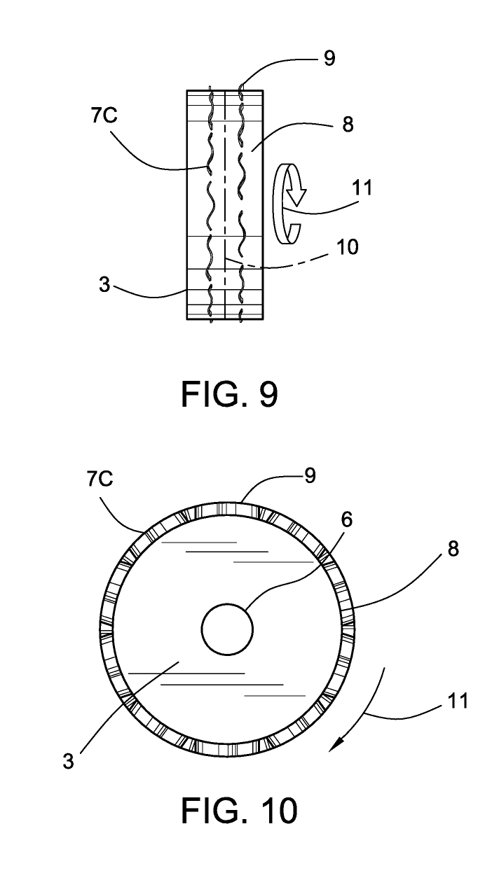

Cutting element 7 may take other shapes. Cutting elements 7B, shown in FIGS. 7-8, and 7C, shown in FIGS. 9-10, may be characterized as S-shaped or as wave-shaped, respectively. In contrast to cutting elements 7A, cutting elements 7B and 7C are an open shape, meaning that the cutting edge 9 of cutting elements 7B and 7C does not close back on itself.

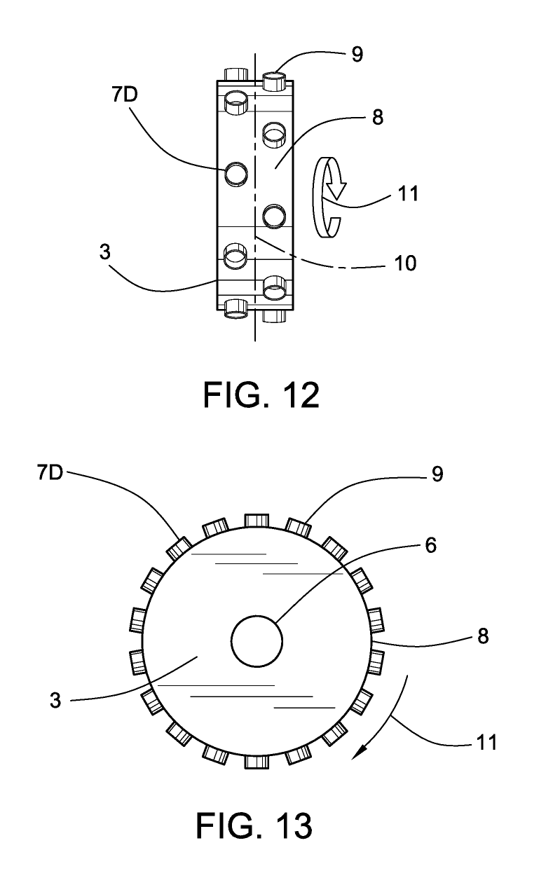

FIGS. 12-13 show cutting element 7D. Like cutting element 7A, cutting element 7D is a closed shape. In particular, cutting element 7D is circular in shape.

A cutting element 7 may be symmetric with respect to one or both of its length and width. For example, cutting elements 7A and 7D are symmetric with respect to both their length and width. In contrast, cutting element 7B is asymmetric with respect to both its length and width, while cutting element 7C is symmetric with respect to its length but asymmetric with respect to its width.

Other configurations of cutting element 7 are possible and within the scope of the invention.

The cutting elements 7 may be arranged in a variety of configurations or patterns. FIGS. 5-13 show several example configurations of cutting elements 7 on cutting wheel 3. The configurations and patterns of cutting elements 7 may be patterned with respect to a center line circumference 10 of cutting wheel 3. For example, when cutting wheel 3 includes a plurality of cutting elements 7, a first cutting element may be disposed on outer periphery 8 to one side of center line circumference 10, and a second cutting element may be disposed on the other side of center line circumference 10, opposite the first cutting element, as are cutting elements 7A and 7B shown in FIGS. 5-8. Cutting elements 7 on opposite sides of center line circumference 10 may be mirror images of each other. As shown with cutting elements 7C in FIGS. 9-10 and cutting elements 7D in FIGS. 12-13, cutting elements 7 may be sequenced so that a cutting element 7 on one side of center line circumference 10 is displaced from a cutting element 7 on the other side of center line circumference 10 along a direction of rotation 11 of cutting wheel 3.

Cutting elements 7 may also be angled with respect to center line circumference 10 of cutting wheel 3. Referring again to FIG. 5, a line drawn through the length of cutting element 7A has an angle of deviation 12 with respect to center line circumference 10. Cutting elements 7B and 7C have a 0° angle of deviation with respect to center line circumference 10.

Two or more rows of cutting elements 7 may be arranged around the outer periphery 8 of cutting wheel 3, as shown most clearly in FIGS. 5, 7, 9, and 11-12. Each row of cutting elements 7 may be displaced from the other rows of cutting elements 7 in a direction orthogonal to center line circumference 10 of cutting wheel 3. Alternatively, cutting wheel 3 could include a single row of cutting elements 7 arranged around outer periphery 8.

The handle 2 of implement 1 is long enough to provide leverage on the implement 1 during use such that the cutting edges 9 of the cutting elements 7 can cut or perforate tissue, but short enough such that the implement 1 can be inserted within the body cavity of a patient. For example, handle 2 may be approximately eight inches in length. Handle 2 may also include one or more channels 13 running along a longitudinal axis of handle 2. Channels 13 increase the ability of a user of implement 1 to grip implement 1, such that the user can impart sufficient leverage on implement 1 to cut or perforate the desired tissue.

In an embodiment, the ratio of the length of handle 2 to the diameter of the cutting wheel 3 ranges from approximately 7:1 to 8:1, and is preferably 7.5:1; the ratio of the length of the handle 2 to the width of the cutting wheel 3 ranges from approximately 20:1 to 30:1, and is preferably 25:1; and the ratio of the diameter of the cutting wheel 3 to its width ranges from approximately 3:1 to 4:1, and is preferably 3.3:1.

Cutting wheel 3 may be disposable and handle 2 may be reusable. For example, cutting wheel 3 may be removed from handle 2 and discarded after implement 1 has been used to increase vascularization of the tissue of a first patient. After sterilization, handle 2 may then be used with a new cutting wheel 3 to increase the vascularization of the tissue of a second patient. The interchangeability of cutting wheel 3 of implement 1 permits a user of implement 1 to select a cutting wheel 3 with a particular cutting element 7, for example the cutting wheels 3 shown in FIGS. 5-13, depending on how the user wants to cut or perforate tissue. In another embodiment, cutting wheel 3 may be reused assuming that cutting wheel 3 is sterilized after use with a first patient.

The components of implement 1 may be made of various materials including metal, such as a surgical stainless steel, plastic, nylon, ceramic, and carbon fiber. The handle 2 and the cutting wheel 3 may be made of the same material or of different materials.

Other variations of implement 1 are contemplated. For example, the rotation of cutting wheel 3 may be motorized such that rotation of the cutting wheel 3 is driven by a motor. Implement 1 could be used by a robot, rather than a human, to increase vascularization in tissue. The cutting wheel 3 may include a ratcheting action, such that the rotation of the cutting wheel 3 is restricted to one direction and/or the rotation of the cutting wheel 3 occurs in discrete steps, for example, eleven ratcheting strokes to complete one full rotation of cutting wheel 3.

A method for increasing vascularization in tissue, such as scar tissue, includes rolling a cutting wheel 3 on the tissue so as to perforate the tissue, wherein the cutting wheel 3 is rotatable around a receiving post 4 attached to a handle 2, the cutting wheel 3 including one or more cutting elements 7 disposed on an outer periphery 8 of the cutting wheel 3.

In order to cut or perforate the tissue, the cutting wheel 3 is mounted on receiving post 4 of handle 2, and retainer 5 is moved from the first position to the second position. The user of implement 1 then places at least one cutting element 7 of cutting wheel 3 on the tissue such that the cutting edge 9 of the at least one cutting element 7 contacts the tissue. The user then imparts a force on implement 1 via handle 2, for example by pushing or pulling, while maintaining contact between the cutting element 7 and the tissue. The force imparted on implement 1 by the user causes the cutting edge 9 of the at least one cutting element 7 to cut or perforate the tissue and also results in the rotation of the cutting wheel 3 so that the cutting edge 9 of another cutting element 7 begins to cut or perforate a different portion of the tissue. When the tissue has been cut or perforated as desired by the user, the user stops imparting a force on implement 1 so that no cutting element 7 contacts the tissue.

A method for applying a growth factor or medication to tissue includes applying the growth factor or medication to one or more cutting elements 7 of the cutting wheel 3, placing the one or more cutting elements 7 on the tissue, and pushing or pulling the handle 2 so as to roll the one or more cutting elements 7 on the tissue.

A method for assembling an implement 1 for increasing vascularization in tissue, such as scar tissue, includes moving a retainer 5 of a handle 2 of the implement 1 to a first position wherein the retainer 5 does not impede the insertion of a receiving post 4 of handle 2 into a bore 6 of a cutting wheel 3, and moving the retainer 5 to a second position wherein the retainer 5 prevents cutting wheel 3 from disengaging from receiving post 4, wherein cutting wheel 3 is freely rotatable about receiving post 4 when mounted thereon.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B, and C” should be interpreted as one or more of a group of elements consisting of A, B, and C, and should not be interpreted as requiring at least one of each of the listed elements A, B, and C, regardless of whether A, B, and C are related as categories or otherwise. Moreover, the recitation of “A, B, and/or C” or “at least one of A, B, or C” should be interpreted as including any singular entity from the listed elements, e.g., A, any subset from the listed elements, e.g., A and B, or the entire list of elements A, B, and C.

### LIST OF REFERENCE SYMBOLS

1 implement
2 handle
3 cutting wheel
4 receiving post
5 retainer
6 bore
7 cutting element
8 outer periphery
9 cutting edge
10 center line circumference
11 direction of rotation
12 angle of deviation
13 channel

1. An implement for increasing tissue vascularization, comprising:
a handle, the handle including a receiving post and a retainer movable between at least a first position and a second position; and
a cutting wheel disposed on the receiving post, the cutting wheel including at least one cutting element disposed on an outer periphery of the cutting wheel,
wherein, in the first position, the handle is configured so as to permit the cutting wheel to be mounted on the receiving post, and
wherein, in the second position, the cutting wheel is held in place with respect to the receiving post yet freely rotatable with respect to the receiving post.

2. The implement for increasing tissue vascularization of claim 1, wherein in the first position the retainer is orthogonal to a longitudinal axis of the handle, and in the second position the retainer is collinear with the longitudinal axis of the handle.

3. The implement for increasing tissue vascularization of claim 1, wherein the cutting wheel further comprises a bore that is complementary in shape to the receiving post.

4. The implement for increasing tissue vascularization of claim 3, wherein both the bore and the receiving post are cylindrical in shape.

5. The implement for increasing tissue vascularization of claim 1, wherein the at least one cutting element comprises a plurality of cutting elements.

6. The implement for increasing tissue vascularization of claim 5, wherein the cutting wheel has a center line circumference and the plurality of cutting elements comprises a first row of cutting elements disposed about the outer periphery of the cutting wheel to a first side of the center line circumference and a second row of cutting elements disposed about the outer periphery of the cutting wheel to a second side of the center line circumference.

7. The implement for increasing tissue vascularization of claim 6, wherein the first row of cutting elements comprises a plurality of cutting elements of a first shape and the second row of cutting elements comprises a plurality of cutting elements of a second shape that is different than the first shape.

8. The implement for increasing tissue vascularization of claim 1, wherein the at least one cutting element is peanut-shaped.

9. The implement for increasing tissue vascularization of claim 1, wherein the at least one cutting element is S-shaped.

10. The implement for increasing tissue vascularization of claim 1, wherein the at least one cutting element is wave-shaped.

11. The implement for increasing tissue vascularization of claim 1, wherein the at least one cutting element is circular.

12. The implement for increasing tissue vascularization of claim 1, wherein the cutting wheel has a center line circumference and the at least one cutting element is angled with respect to the center line circumference.

13. The implement for increasing tissue vascularization of claim 1, wherein the at least one cutting element protrudes radially outward from the outer periphery of the cutting wheel.

14. The implement for increasing tissue vascularization of claim 1, wherein a portion of the at least one cutting element farthest from the outer periphery of the cutting wheel comprises a cutting edge.

15. The implement for increasing tissue vascularization of claim 14, wherein a height of the at least one cutting element is the distance from the outer periphery of the cutting wheel to the cutting edge, and the height ranges from 1 to 5 mm.

16. The implement for increasing tissue vascularization of claim 1, wherein a ratio of a length of the handle to a diameter of the cutting wheel ranges from approximately 7:1 to 8:1, a ratio of the length of the handle to a width of the cutting wheel ranges from approximately 20:1 to 30:1, and a ratio of the diameter of the cutting wheel to its width ranges from approximately 3:1 to 4:1.

17. The implement for increasing tissue vascularization of claim 1, further comprising at least one channel running along a longitudinal axis of the handle.

18. The implement for increasing tissue vascularization of claim 1, wherein the cutting wheel is at least one of disposable or reusable.

19. The implement for increasing tissue vascularization of claim 1, wherein the at least one cutting element is symmetric with respect to both its length and its width.

20. A method for increasing tissue vascularization using the implement of claim 1, the method comprising:
moving the retainer from the first position to the second position;
placing the at least one cutting element on the tissue; and
pushing or pulling the handle so as to roll the cutting wheel on the tissue.

21. A method for applying a growth factor or medication to tissue using the implement of claim 1, the method comprising:
applying at least one of the growth factor or the medication to the at least one cutting element;
placing the at least one cutting element on the tissue; and
pushing or pulling the handle so as to roll the at least one cutting element on the tissue.

22. A method for assembling an implement for increasing vascularization, the implement including a handle with a retainer and a receiving post, and a cutting wheel that includes at least one cutting element disposed on an outer periphery of the cutting wheel, the method comprising:
moving the retainer to a first position so as to permit the cutting wheel to be mounted on the receiving post;
mounting the cutting wheel on the receiving post; and
moving the retainer to a second position wherein the cutting wheel is held in place with respect to the receiving post yet freely rotatable with respect to the receiving post.
